(19) United States
(12) Patent Application Publication
(10) Pub. No.: US 2014/0308221 A1
(43) Pub. Date: Oct. 16, 2014

(54) POROUS AND/OR HOLLOW MATERIAL CONTAINING UV ATTENUATING NANOPARTICLES, METHOD OF PRODUCTION AND USE

(71) Applicants: David Schlossman, Short Hills, NJ (US); Yun Shao, South Plainfield, NJ (US); Frank Mazzella, Franklin Park, NJ (US)

(72) Inventors: David Schlossman, Short Hills, NJ (US); Yun Shao, South Plainfield, NJ (US); Frank Mazzella, Franklin Park, NJ (US)

(73) Assignee: KOBO PRODUCTS, INC., South Plainfield, NJ (US)

(21) Appl. No.: 14/229,818

(22) Filed: Mar. 28, 2014

Related U.S. Application Data
(62) Division of application No. 12/403,431, filed on Mar. 13, 2009, now abandoned.
(60) Provisional application No. 61/044,274, filed on Apr. 11, 2008.

(51) Int. Cl. A61K 8/02; A61K 8/29; A61Q 17/04; A61K 8/28; A61K 8/19; A61K 8/25; A61K 8/27; A61K 8/26

(52) U.S. Cl. CPC A61K 8/0279; A61K 8/27; A61K 8/29; A61K 8/26; A61K 8/28; A61K 8/19; A61K 8/25; A61Q 17/04
USPC 424/59; 427/160

(57) ABSTRACT

The present invention provides UV attenuating nanoparticles entrapped in porous particulates that are coated with a wax material. The porous particulates also include a fatty acid applied to the wax coating. Also provided is a method of producing a powder comprised of UV attenuating nanoparticles entrapped in porous particulates coated with a wax material. Further provided is a composition, such as a cosmetic composition, which includes the porous particulates loaded with the UV attenuating nanoparticles.

### FIELD OF THE INVENTION

The present invention relates generally to powders, comprising, for example nanoparticles (or at least very small particulates) contained within a porous and/or hollow material so that the nanoparticles do not diffuse onto and penetrate into the skin if topically applied, for use in cosmetic and other over-the-counter compositions. More particularly, the powders disclosed herein relate to novel particulates having UV attenuating nanoparticles contained in voids of the particulates, a method for producing such powders, and formulations that include such powders.

### BACKGROUND

Inorganic UV filters such as titanium dioxide and zinc oxide have been used globally as sunscreen agents for over twenty years to prevent sun-caused damage, which can range from irritation to premature “aging” and skin cancer. Zinc oxide and titanium dioxide are substantially hypoallergenic and, in use, unlike organic sunscreens, are far less likely to cause adverse reactions. Furthermore, their stability compared to organic sunscreens is a substantial additional asset.

However, titanium dioxide and zinc oxide can cause undesired whitening on skin when their particle size is too large. To improve the aesthetics of suncare products containing inorganic materials such as titanium dioxide and zinc oxide, microparticles of titanium dioxide and zinc oxide have been developed. They are transparent on the skin and aesthetically appealing and are in extensive use today. These micro grades typically have primary particle sizes of less than 100 nm when analyzed using TEM. Particles less than 100 nm are often referred to as nanoparticles.

Many other materials used in personal care products also contain so-called nanoparticles. For example, transparent oxides are used in color cosmetics and carbon black (Black No. 2) is commonly in mascara and eyeliners.

Recently, some have speculated that nanoparticles may have some unspecified adverse effect, despite the non-existence of any supporting clinical data. There is a perception among some people that these fine particles could penetrate the skin and cause harm to human health. Thus, these products in recent years have come under heightened scrutiny.

In view of the perceived health risk associated with nanoparticles, pigment producers have been challenged recently to produce particles and/or composite powders with particles that are all or almost all larger than 100 nm, and preferably larger than 150 nm, as measured by TEM. However, such large particles in a sunscreen tend to make the skin appear chalky and unattractive. Moreover, larger particles do not provide the degree of protection against ultraviolet light desirably achieved in a sunscreen. A nanoparticle which is not small would be useful in sunscreens, cosmetics and other over-the-counter compositions which can provide protection against UV light while maintaining a natural, transparent and attractive appearance upon application to skin and other surfaces. However, larger particles do not have desirable optical properties.

### SUMMARY OF THE INVENTION

The present invention achieves smaller particle optics in large particles through the use of coating or other expedients as described herein, for example by providing powder compositions and methods to produce powder compositions, more particularly composite powders, for inclusion in cosmetic and other over-the-counter compositions. The inventive products comprise micronized UV attenuating particles, also referred to herein as, UV attenuating nanoparticles, contained within pores, hollow portions, or other voids of “porous” particulates to keep the nanoparticles from direct contact with the surface it is applied to, for example, skin. The composite powder has all or almost all of the porous particulates contained therein loaded with the nanoparticles, in which the particulate size is larger than 100 nm. The composite powders can be incorporated into dispersions or compositions as larger-sized sunscreen solids.

In an aspect of the present invention, there is provided a powder, comprised of a plurality of porous particulates, a plurality of ultraviolet (UV) attenuating nanoparticles entrapped in each of the porous particulates, and a wax material coated on each of the plurality of the porous particulates.

In another aspect of the present invention, there is provided a method of producing a powder, comprised of combining a plurality of particulates, the particulates having at least one void therein, with a plurality of UV attenuating nanoparticles, so that the UV attenuating nanoparticles enter voids in the plurality of particulates to form nanoparticle-particulate composites. Wax is added at a temperature above the melting point of the wax to the nanoparticle-particulate composites. The melted wax is mixed with the nanoparticle-particulate composites.

### DETAILED DESCRIPTION

The present invention is directed to a nanoparticle-particulate composite powder, comprised of UV attenuating nanoparticles absorbed in porous particulates in a form that keeps the nanoparticles from coming into unimpeded direct contact with skin. The UV attenuating nanoparticles can be used beneficially in cosmetic compositions and over-the-counter drug and other product compositions without concern for possible penetration and/or unspecified adverse effects regarding the use of such nanoparticles on skin. The powder may include a hydrophobic coating useful in incorporating the powder into oil-based compositions. The present invention also is directed to a method for preparing the UV attenuating nanoparticle-particulate composites and the coated powders resulting therefrom. The present invention extends to products that incorporate the UV attenuating nanoparticle-particulate composite powders, such as novel cosmetic compositions which include the coated powders of the present invention.

As used herein, the terms “nanoparticles” and “micronized particles” are interchangeable and include a material having 5% or more of the nanoparticles, in which the nanoparticles have a size less than about 100 nm, for example 50-150 nm.

As used herein, the terms “particulates” and “particles” are interchangeable, and refer to particles having a size greater than about 100 nm, for example 50-150 nm.

The entrapped property of the UV attenuating nanoparticles in the nanoparticle-particulate composite powder makes the powder especially attractive to formulators in the cosmetics industry, allowing for these powders to be used for a wide range of applications without undue concern regarding possible adverse effects due to the nanoparticles contacting skin or other surfaces to which it is applied. Thus, the formulator may freely incorporate the UV attenuating nanoparticle-particulate composites of the present invention fabricated from substances such as metal oxides, dyes, and carbon black into cosmetic compositions to meet an exceptionally diversified range of cosmetics requirements.

The powder of the present invention provides all the benefits of using nanoparticles such as attenuation of UV light, good transparency, good skin feel and reduced skin whitening, without placing the nanoparticles into unimpeded direct contact with the skin or other surface. The powder also may include a hydrophobic coating to achieve good dispersion stability and to improve its properties to make desirable sunscreens and cosmetics.

The powder of the present invention may be formulated into a dispersion that is incorporated into a composition such as a cosmetic composition or a sunscreen. The cosmetic composition may be a liquid or dry make-up such as foundation or pressed powder, lipstick, blush, eyeshadow, or mascara. Additionally, the cosmetic composition may be anhydrous or an emulsion.

In one embodiment, the powder of the present invention comprises a particulate having voids filled with UV attenuating nanoparticles and coated with a wax to contain the nanoparticles within the voids. The powder may include a fatty acid applied to the wax coating. The resulting composite particulates of the powder, with or without the presence of a fatty acid, have all or substantially all particulate sizes greater than 100 nm.

UV attenuating nanoparticles suitable for entrapment in the voids of the particulate include any UV attenuating nanoparticles that are capable of entering the voids as a powder or as liquid. The liquid containing the UV attenuating nanoparticles may be a solution, suspension, dispersion, or colloid. The nanoparticles may have any desired regular or irregular shape including spherical or ball like nanoparticles with irregular porous surfaces, needles, rods, flakes, rhomboids, nodular, acicular, granular, ellipsoidal, hexagonal, prismatic, star-like, Y-shaped, and the like, but with nanoparticle sizes less than 100 nm.

The UV attenuating nanoparticles are comprised of inorganic pigments, dyes, and mixtures thereof. Suitable inorganic pigments may include, without limitation, titanium dioxide; zinc oxide; zirconium oxide; iron oxides; aluminum oxide; chromium oxide; cerium oxide; manganese; clear plastics; high index of refraction glass; violet; ultramarines, composites of metal oxides or of a metal oxide and an inorganic salt and any other inorganic pigment powder useful in the cosmetic or other relevant arts.

In one embodiment, the metal oxide particles may be coated with oxides of other elements such as oxides of aluminium, zirconium or silicon, or mixtures thereof such as alumina and silica as disclosed in GB-2205088-A, the teaching of which is incorporated herein by reference. Alternately, the nanoparticles may be treated with other known inorganic coatings, singly or in combination, before incorporation into the voids of the particulate. The amount of inorganic coating is in the range of about 2% to about 25%, preferably from about 4% to about 20%, more preferably from about 6% to about 15%, and especially from about 8% to about 12% by weight, calculated with respect to the weight of the UV attenuating nanoparticles. The inorganic coating may be applied using techniques known in the art. The inorganic coating, if present, is preferably applied as a first layer to the surface of the metal oxide.

In one embodiment, the powders of the present invention may include an organic coating that gives the pigments hydrophobic properties. The organic coating may be applied to the inorganic coating. The hydrophobic coating agent may be, for example, a silicone, a silane, a metal soap, a titanate, an organic wax, and mixtures thereof. Alternatively, the hydrophobic coating may include a fatty acid, for example, a fatty acid containing 10 to 20 carbon atoms, such as lauric acid, stearic acid, isostearic acid, and salts of these fatty acids. The fatty acid may be isopropyl titanium trisostearate. With respect to the silicone, the hydrophobic coating may be a methicone, a dimethicone, their copolymers or mixtures thereof. The silicone may also be an organosilicon compound, for example dimethylpolysiloxanes having a backbone of repeating—Me2SiO—units (“Me” is methyl, CH₃), methyl hydrogen polysiloxanes having a backbone of repeating—MeHSiO—units and alkoxysilanes of formula RnOSiH(₄₋ₙ) where “R” is alkyl and “n” is the integer 1, 2 or 3. With respect to the silane, the hydrophobic coating agent may be an alkoxysilane, for example an alkyltriethoxy or an alkyltrimethoxy silane available from OSI Specialties or PCR. The alkoxysilane may be a triethoxycaprylylsilane or a perfluoroalkylethyl triethoxysilane having a C3 to C12 alkyl group that is straight or branched. One such alkoxysilane is Dynasylan® OCTEO available from Degussa AG. With respect to the metal soap, the hydrophobic coating agent may be a metal myristate, metal stearate, a metal palmitate, a metal laurate or other fatty acid derivatives known to those skilled in the art. The metal, for example, may be magnesium or aluminum. With respect to the titanate, the hydrophobic coating agent may be an organotitanate as taught in U.S. Pat. No. 4,877,604 to Mitchell Schlossman (hereinafter “Schlossman '604”), the disclosure of which is herein incorporated by reference. Schlossman '604 discloses isopropyl titanium triisostearate as one preferred coating agent. With respect to the organic wax, the hydrophobic coating agent may be a synthetic wax like polyethylene or a natural wax like carnauba wax.

In one embodiment, the powders of the present invention may include an organic coating that gives the pigments hydrophilic properties. The organic coating may be applied to the inorganic coating. The hydrophobic coating agent may be, for example, PEG-9 methylether triethoxysilane, PEG-12 dimethicone, sodium alginate, and polysaccharide or its derivatives.

In one embodiment, the metal oxide nanoparticles are coated with both an inorganic and an organic coating, either sequentially or as a mixture. It is preferred that the inorganic coating, preferably alumina, is applied first followed by the organic coating, preferably any of the hydrophobic coatings discussed above.

Suitable dyes include lakes of calcium, barium, aluminum or zirconium salts of FD&C and D&C grades of Red No. 6, Red No. 7, Red 21, Red No. 27 and Yellow No. 5. Other suitable pigments include ferric blue, carbon black. Other suitable UV attenuating nanoparticles are known or will become apparent to those skilled in the art.

Particulates having a void for use in formulating the powder of the present invention may be inorganic or organic. As used herein, voids may include pores, crevices, cavities, hollow portions, or the like formed in the particulate. The porous particulates of the present invention contain entrapped UV attenuating nanoparticles to provide a suitable powder for use in cosmetic or over-the-counter compositions. The pore or pores may be completely enclosed or encapsulated by the particulate material or may be partially enclosed and open to the surface of the particulate. The porous particulate may have a single pore which is partially enclosed by a solid shell or a plurality of pores. A plurality of pores may be interconnected and may connect to an opening at the surface of the particulate. The particulates may also contain pores which are completely enclosed and are not interconnected or open to the surface of the particulate. Particulates with non-interconnected and completely enclosed pores are known as closed cell foam type particles. Thus, the particulates are porous or hollow and can contain an entrapped or partially entrapped UV attenuating nanoparticle in the pore or pores as disclosed herein.

Inorganic particulate material useful in the present invention may exist in an amorphous or glass state or in a crystalline state or in a mixture of amorphous and crystalline forms. The inorganic material useful in this invention includes borates, alumina, carbonates, bicarbonates, silicas, silicates, aluminosilicates, and phosphates in the form of monomeric salts or as polymeric or condensed forms, or as mixtures of monomeric and polymeric forms. Particulates comprising mixtures of these materials are also expected to be useful in the present invention. Inorganic materials useful in the present invention include, but are not limited to, SiO₂, alkali salts of CO₃²⁻ and HCO₃¹⁻, alkali salts of HPO₄²⁻, aluminum oxides and hydroxides, such as Al₂O₃, alkali salts of aluminosilicates, and H₃BO₃, as taught in Glajch U.S. Pat. No. 5,147,631, the teachings of which are incorporated herein by reference.

Silicates and silicas, as used herein, include any and all siliceous materials in the particulate form stated above. Typical silica materials include SiO₂, silicate-containing minerals, and synthetic silicates such as silica gels, powders, porous glass and those prepared by hydrolysis of calcium silicide or sodium silicate. The preparation of porous silica particles is described in Bergna and Kirkland, U.S. Pat. No. 4,131,542, Kirkland, U.S. Pat. No. 3,782,075, and Kirkland, U.S. Pat. No. 3,505,785, the teaching of which is incorporated herein by reference. Silica also is commercially available as porous spherical silica beads such as MSS-500 and MSS-500/3H from Kobo Products, Inc.

The inorganic particulates of the invention have the advantage of good mechanical stability and rigidity, which are important attributes. In addition, inorganic particulates can be prepared and fabricated, using known techniques, into a variety of shapes, sizes, and degrees of porosity, in order to obtain the most desirable UV attenuating nanoparticle loading. The inorganic particulates are capable of absorbing liquid, especially the pores or hollows therein, so the UV attenuating nanoparticles can be loaded as a liquid solution, suspension, dispersion, colloid, and the like.

The inorganic particles useful in the present invention may range in size and shape or morphology. A variety of particle shapes are useful in the present invention. For example, the particles may range from roughly spherical shapes to rod-like shapes and may be regular or irregular, flat or granular in shape. The particle size, measured as the average particle diameter, should be in the range of about 3 microns to 50 microns. For irregular shaped particles, the term average particle diameter refers to the effective particle diameter or Stokes diameter of the particle.

Likewise, organic particulates of similar shapes, sizes and voids, are useful for entrapping the UV attenuating powders of the invention. The organic porous material useful in this invention includes, without limitation, polyethylene, polypropylene, ethylene-vinyl acetate copolymer, polystyrene, styreneacrylamide copolymer and the like, cellulose and other naturally occurring bodies, cellulose acetate, nylon, polyester, polymethylmethacrylate, and other porous synthetic resins, alone or in combination. Organic porous particulates also are commercially available such as the Poly-pore® series with the INCI name allyl methacrylates crosspolymer.

Wax for coating the porous particulates to contain the nanoparticles within the voids of the particulates may include, without limitation, a natural wax, a synthetic wax, and mixtures thereof. As used herein, “wax” refers to a natural or synthetic material having the following characteristics: it is essentially non-water soluble (i.e., <5%); it has a melting point preferably below 100° C. but not above 200° C.; and it has a viscosity of less than 500 cp at a temperature less than 100° C. In one embodiment, the “wax” may be a combination of substances that together possess these characteristics in combination. Waxes include, but are not limited to, natural and synthetic waxes that contain mixtures of alkyl wax esters, resins, and other vegetable matter components; clay-treated microcrystalline waxes; oxidized hydrocarbon waxes; natural and synthetic beeswax, auto-oxidized beeswax, candelilia, carnauba, and synthetic waxes prepared by esterification of natural plant-derived fatty acids and alcohols; various grades of paraffin waxes; and natural and synthetic oils. Synthetic waxes may include ethene homopolymers, such as polyethylene.

Fatty acids for application to the wax layer or coating include lauric acid, myristic acid, palmitic acid, stearic acid, and derivatives thereof, alone or in combination. The fatty acid may bond covalently to a reactive moiety on the wax layer or may have ionic, hydrogen, or van der Waals bonding in addition to, or alternatively to, the covalent bonding, to provide satisfactory bonding or connection between the fatty acid layer and the wax layer.

The powder of the present invention having wax coated porous particulates containing UV attenuating nanoparticles in its pores may include the following percent by weight of the UV attenuating nanoparticles, porous particulates, wax, and fatty acid.

TABLE 1

| Substance | Percent by weight |
|---|---|
| UV attenuating nanoparticles | 10-80% |
| Particulate (porous/hollow) | 20-80% |
| Wax | 1-30% |
| Fatty Acid (optional) | 1-15% |

The wax coated nanoparticle-particulate composites may be formulated according to the following process. A plurality of porous particulates, in which porous particulates have at least one void therein is combined with a plurality of UV attenuating nanoparticles so that the nanoparticles enter the voids of the porous particulates. The porous particulates and UV attenuating nanoparticles may be combined in at least one of two methods. The first method includes adding a dispersion of UV attenuating nanoparticles under agitation to the porous particulates until the dispersion is absorbed by the particulates. Dispersion of the UV attenuating nanoparticles may be performed according to known methods familiar to those skilled in the art. If the solvent used to make the dispersion is a volatile solvent, the nanoparticle-particulate composite may be heated (under vacuum) to remove the solvent at temperatures commonly used to remove solvents and known by those skilled in the solvent art for the particular solvent. Additional amounts of the dispersion may be added under agitation to the porous particulate, repeatedly, to achieve maximum loading of the nanoparticles in the voids of the particulate. Alternately, dry UV attenuating nanoparticles may be blended with the porous particulates until the nanoparticles enter the voids of the particulates. This method also may be repeated for maximum loading of the nanoparticles.

Once the UV attenuating nanoparticles are loaded into the particulates, by either method, wax is added with mixing or blending at a temperature above the melting point of the wax. The nanoparticle-particulate composite is preferably a dry powder when the wax is added and is mixed or blended until the wax is uniformly distributed. The wax coats the particulates and entraps the nanoparticles within the void so the nanoparticles are prevented from exiting the particles, which in topical applications keeps the nanoparticles from contacting the skin.

A fatty acid may be added to the wax coated particulates. The fatty acid may be applied according to known methods to those skilled in the art. Preferably, the fatty acid is applied to the wax-coated particulates at a temperature above the melting point of the fatty acid with mixing or blending until uniformly distributed. Alternately, the fatty acid can be applied at a temperature above the melting point of the wax with mixing or blending until uniformly distributed. In this embodiment, the wax and fatty acid may mix to form a combination wax-fatty acid coating on the particulates. In another embodiment, the fatty acid can be sprayed onto the wax-coated particulates as a solution, dispersion, colloid, or suspension. Suitable solvents for a fatty acid solution include, without limitation, alcohols such as isopropanol, acetones and alkanes. The solvent is then removed by heat under vacuum.

The nanoparticle-particulate composites, after being mixed with the wax and optionally the fatty acid, are cooled to room temperature. The cooled particulates form a dry powder that contains entrapped UV attenuating nanoparticles. The powder may be milled to break up any lumps within the powder and is preferably free of oversized particles that may impart grittiness.

In another aspect, the nanoparticle-particulate composites may be incorporated into a slurry, or preferably a liquid dispersion using known techniques familiar to those skilled in the art. The dispersing medium may be any suitable aqueous or organic liquid medium.

Cosmetically acceptable materials are preferred as the liquid medium. A useful organic medium are liquid oils such as vegetable oils, e.g. fatty acid glycerides, fatty acid esters and fatty alcohols. Another preferred medium is a siloxane fluid, especially a cyclic oligomeric dialkylsiloxane, such as the cyclic pentamer of dimethylsiloxane known as cyclomethicone. Alternative fluids include dimethylsiloxane linear oligomers or polymers having a suitable fluidity and phenyltris(trimethylsiloxy)silane (also known as phenyltrimethicone). Another preferred organic medium is a silicone fluid, for example methicone, dimethicone, other silicone derivatives, and combinations thereof.

Examples of suitable organic media include, without limitation, avocado oil, C12-15 alkyl benzoate, C12-15 alkyl ethylhexanoate, C12-15 alkyl lactate, C12-15 alkyl salicylate, C13-14 isoparaffin, C18-36 acid glycol ester, C18-36 acid triglyceride, caprylic/capric glycerides, caprylic/capric triglyceride, caprylic/capric/lauric triglyceride, caprylic/capric/linoleic triglyceride, caprylic/capric/myristic/stearic triglyceride, caprylic/capric/stearic triglyceride, castor oil, castor oil-silicone ester, cetearyl ethylhexanoate, cetearyl isononanoate, cetearyl palmitate, cetearyl stearate, cetyl dimethicone, cetyl dimethicone copolyol, cetyl ethylhexanoate, cetyl glycol isostearate, cetyl isononanoate, cetyl lactate, cetyl myristate, cetyl oleate, cetyl palmitate, cetyl ricinoleate, cetyl stearate, cocoglycerides, coconut oil, cyclomethicone, cyclopentasiloxane, cyclotetrasiloxane, decyl isostearate, decyl oleate, decyl polyglucoside, dibutyl adipate, diethylhexyl dimer dilinoleate, diethylhexyl malate, diisopropyl adipate, diisopropyl dimer dilinoleate, diisostearoyl trimethylolpropane siloxy silicate, diisostearyl adipate, diisostearyl dimer dilinoleate, diisostearyl malate, diisostearyl trimethylolpropane siloxy silicate, dilauroyl trimethylolpropane siloxy silicate, dilauryl trimethylolpropane siloxy silicate, dimethicone, dimethicone copolyol, dimethicone propyl PG-betaine, dimethiconol, dimethyl isosorbide, dioctyl maleate, dioctylodedecyl dimer dilonoleate, ethylhexyl benzoate, ethylhexyl cocoate, ethylhexyl dimethyl PABA, ethylhexyl ethylhexanoate, ethylhexyl hydroxystearate, ethylhexyl hydroxystearate benzoate, ethylhexyl isononanoate, ethylhexyl isopalmitate, ethylhexyl isostearate, ethylhexyl laurate, ethylhexyl methoxycinnamate, ethylhexyl myristate, ethylhexyl neopentanoate, ethylhexyl oleate, ethylhexyl palmitate, ethylhexyl salicylate, ethylhexyl stearate, glyceryl caprate, glyceryl caprylate, glyceryl caprylate/caprate, glyceryl cocoate, glyceryl dilaurate, glyceryl dioleate, glyceryl hydroxystearate, glyceryl isostearate, glyceryl laurate, glyceryl oleate, glycol oleate, glycol ricinoleate, helianthus annuus (hybrid sunflower) seed oil, helianthus annuus (sunflower) seed oil, homosalate, isoamyl laurate, isoamyl p-methoxycinnamate, isocetyl alcohol, isocetyl behenate, isocetyl ethylhexanoate, isocetyl isostearate, isocetyl laurate, isocetyl linoleoyl stearate, isocetyl myristate, isocetyl palmitate, isocetyl salicylate, isocetyl stearate, isocetyl stearoyl stearate, isohexadecane, isononyl isononanoate, isopropyl C12-15-pareth-9 carboxylate, isopropyl isostearate, isopropyl lanolate, isopropyl laurate, isopropyl linoleate, isopropyl methoxycinnamate, isopropyl myristate, isopropyl oleate, isopropyl palmitate, isopropyl PPG-2-isodeceth-7 carboxylate, isopropyl ricinoleate, isopropyl stearate, isostearic acid, isostearyl alcohol, isostearyl ethylhexanoate, isostearyl isononanoate, isostearyl isostearate, isostearyl lactate, isostearyl myristate, isostearyl neopentanoate, isostearyl palmitate, isostearyl stearoyl stearate, jojoba oil, lanolin (lanolin oil), maleated soybean oil, myristyl isostearate, myristyl lactate, myristyl myristate, myristyl neopentanoate, myristyl stearate, octocrylene, octyldecanol, octyldodecanol, oenothera biennis (evening primrose oil), paraffinum liquidum (mineral oil), PCA dimethicone, pentaerythrityl tetraisononanoate, pentaerythrityl tetraisostearate, perfluoropolymethylisopropyl ether, persea gratissima (avocado oil), phenyl trimethicone, PPG-15 stearyl ether, propylene glycol ceteth-3 acetate, propylene glycol dicaprylate, propylene glycol dicaprylate/dicaprate, propylene glycol dipelargonate, propylene glycol distearate, propylene glycol isoceteth-3 acetate, propylene glycol isostearate, propylene glycol laurate, proylene glycol ricinoleate, propylene glycol stearate, prunus dulcis (sweet almond oil), squalane, squalene, tricaprylin, tricaprylyl citrate, tridecyl ethylhexanoate, tridecyl neopentanoate, tridecyl stearoyl stearate, triethylhexanoin, triethylhexyl citrate, trihydroxystearin, triisocetyl citrate, triisostearin, triisostearyl citrate, trimethylolpropane triisostearate, trimethylsiloxysilicate, triticum vulgare (wheat germ oil), vitis vinifera (grape) seed oil, and mixtures thereof.

The dispersion containing the nanoparticle-particulate composites may also contain a dispersing agent in order to improve the properties thereof. The dispersing agent is present in the range of about 1% to about 50%, preferably from about 3% to 30%, more preferably from about 5% to about 20%, and especially from about 8% to about 15% by weight based on the total weight of the UV attenuating nanoparticles present.

Suitable dispersing agents for use in an organic medium include, without limitation, substituted carboxylic acids, soap bases and polyhydroxy acids. Typically, the dispersing agent can be one having a formula X.CO.AR in which A is a divalent bridging group, R is a primary secondary or tertiary amino group or a salt thereof with an acid or a quaternary ammonium salt group and X is the residue of a polyester chain, which together with the —CO— group is derived from a hydroxy carboxylic acid of the formula HO—R′—COOH. As examples of typical dispersing agents are those based on ricinoleic acid, hydroxystearic acid, hydrogenated castor oil fatty acid which contains in addition to 12-hydroxystearic acid small amounts of stearic acid and palmitic acid. Dispersing agents based on one or more polyesters or salts of a hydroxycarboxylic acid and a carboxylic acid free of hydroxy groups can also be used. Compounds of various molecular weights can be used. Other suitable dispersing agents are those monoesters of fatty acid alkanolamides and carboxylic acids and their salts. Alkanolamides are based on ethanolamine, propanolamine or aminoethyl ethanolamine for example. Alternative dispersing agents are those based on polymers or copolymers of acrylic or methacrylic acids, e.g. block copolymers of such monomers. Other dispersing agents of similar general form are those having epoxy groups in the constituent radicals such as those based on the ethoxylated phosphate esters. The dispersing agent can be one of those commercially referred to as a hyper dispersant. Suitable dispersing agents for use in an aqueous medium include a polymeric acrylic acid or a salt thereof. Partially or fully neutralized salts are usable e.g. the alkali metal salts and ammonium salts. Examples of dispersing agents are polyacrylic acids, substituted acrylic acid polymers, acrylic copolymers, sodium and/or ammonium salts of polyacrylic acids and sodium and/or ammonium salts of acrylic copolymers. Such dispersing agents are typified by polyacrylic acid itself and sodium or ammonium salts thereof as well as copolymers of an acrylic acid with other suitable monomers such as a sulphonic acid derivative such as 2-acrylamido 2-methyl propane sulphonic acid. Comonomers polymerisable with the acrylic or a substituted acrylic acid can also be one containing a carboxyl grouping. Usually, the dispersing agents have a molecular weight of from 1,000 to 10,000 and are substantially linear molecules.

In another aspect, the powders and/or dispersions of the powders of the present invention may be incorporated into a cosmetic composition. The cosmetic compositions may be anhydrous or emulsions. Examples of cosmetic compositions in which the powders may be employed include liquid or dry make-ups such as foundation or pressed powder, lipsticks, blushes, eyeshadow, and mascara. The nanoparticle-particulate composites and the powder including such composites are beneficial in cosmetic compositions in that the powders entrap the nanoparticles and keep them from contacting skin while still allowing the beneficial properties of the nanoparticles to be used in the compositions, such as attenuating UV light while being transparent to visible light and reduced skin whitening.

Alternatively, the nanoparticle-particulate composites may be incorporated in the form of a lotion or cream of a solid and/or semi-solid dispersion. Suitable solid or semi-solid dispersions may contain, for example, from about 50% to about 90%, preferably from about 60% to about 85% by weight of the nanoparticle-particulate composites of the present invention, together with any one or more of a liquid medium disclosed herein, or a high molecular polymeric material, such as a wax.

The nanoparticle-particulate composite coated powders and dispersions of the present invention are useful as ingredients for preparing sunscreen compositions and sunscreening cosmetics of all types, especially in the form of emulsions. The emulsion may be an oil-in-water, water-in-oil, or a water-in-silicon emulsion. The dispersion may further contain conventional additives suitable for use in the intended application, such as conventional cosmetic ingredients used in sunscreens. Because the UV attenuating nanoparticles attenuate ultraviolet light, a sunscreen composition may include other sunscreen agents, such as organic materials. Suitable organic sunscreens include, without limitation, p-methoxy cinnamic acid esters, salicylic acid esters, p-amino benzoic acid esters, non-sulphonated benzophenone derivatives, derivatives of dibenzoyl methane and esters of 2-cyanoacrylic acid. Specific examples of useful organic sunscreens include benzophenone-1, benzophenone-2, benzophenone-3, benzophenone-6, benzophenone-8, benzophenone-12, isopropyl dibenzoyl methane, butyl methoxy dibenzoyl methane, ethyl dihydroxypropyl PABA, glyceryl PABA, octyl dimethyl PABA, octyl methoxycinnamate, homosalate, octyl salicylate, octyl triazone, octocrylene, etocrylene, menthyl anthranilate, and 4-methylbenzylidene camphor.

Many other products that may benefit from such a versatile coated powder are known to those skilled in the art. The coated powders, for example, may be incorporated into other industrial products where the particle material is customarily used and where hydrophobic and lipophobic properties are beneficial, for example, in paints and plastics.

The present invention is more particularly described in the following non-limiting examples, which are intended to be illustrative only, as numerous modifications and variations therein will be apparent to those skilled in the art.

### Example 1

A novel powder that has the UV attenuating nanoparticles entrapped therein was prepared with the following percent by weight of the substances in Table 2.

TABLE 2

| Constituent | Substance | % by Weight |
|---|---|---|
| UV attenuating nanoparticle | TiO₂ (methicone coated) | 62% |
| dispersion | | |
| Porous Particulate | Porous silica beads | 25% |
| Wax | Carnauba wax | 5% |
| Fatty Acid | Lauric acid | 5% |
| Dispersant | Polyhydroxystearic acid | 3% |

500 mL of a dispersion of 15 nm TiO₂ nanoparticles (commercially available from Kobo Products, Inc. under the trade name PM9P50M170) was added under agitation to 100 g of porous silica beads (commercially available from Kobo Products, Inc. under the trade name Silica Shells). The dispersion of TiO₂ nanoparticles was mixed under agitation with the porous silica beads for 15 min until the dispersion was absorbed by the particulate as evidenced by the mixture becoming a dry powder. The powder was heated at 100° C. under vacuum till the weight was constant. Carnauba wax was heated above its melting point to 110° C. to liquefy the wax and the liquid wax was added into the dry powder of porous silica beads loaded with the TiO₂ nanoparticles. The wax and powder were blended for 1 hour until the wax was uniformly distributed. Then, lauric acid was heated above its melting point to 110° C. and added to the wax coated powder with blending for 1 hour until uniformly distributed in the powder. The powder mixture was cooled to room temperature and thereafter milled to break up any lumps.

### Example 2

Another novel powder was prepared with the following percent by weight of the substances in Table 3.

TABLE 3

| Constituent | Substance | % by Weight |
|---|---|---|
| UV attenuating particles | Carbon black | 20% |
| Porous Particulate | Nylon | 65% |
| Wax | Polyethylene wax | 10% |
| Fatty Acid | Lauric acid | 5% |

20 g of carbon black nanoparticles in powder form was blended with the porous nylon (Kobo Nylon 12 or Nylon 6 Microspheres) for 1 hour until the nanoparticles entered the voids of the particulate. Polyethylene wax (Kobo polyethylene and microcrystalline was PM WAX 82) was heated above its melting point to 120° C. to liquefy the wax and the liquid wax was poured into the dry powder of porous nylon loaded with the carbon black. The wax and powder were blended for 1 hour until the wax was uniformly distributed. Then, lauric acid was heated above its melting point to 110° C. and added to the wax coated powder with blending for 1 hour until uniformly distributed in the powder. The powder mixture then was cooled to room temperature and thereafter milled to break up any lumps.

### Example 3

### Preparation of a Crème to Powder Foundation Incorporating the Novel UV Attenuating Nanoparticles Entrapped in Porous Silica

A crème to powder foundation cosmetic composition, with an SPF of 40.93 including porous silica entrapped TiO₂ prepared as in example 1 (to be released under Kobo 5555M170-CWL5), was prepared to incorporate the UV attenuating void-filled powder of Example 1. The metal oxide powder was first formulated into a dispersion and was then incorporated into a crème to powder foundation cosmetic composition. The following ingredients listed in Table 4 were employed in the proportions indicated to prepare the crème to powder foundation

TABLE 4
Creme to Powder Foundation containing porous silica entrapped
TiO₂ (SS55M170-CWL5)

| | INCI | % by wt |
|---|---|---|
| Part 1 | | |
| Wickenol 155 | Ethylhexyl Palmitate | 36.64 |
| Squalane NF | Squalane | 7.92 |
| Lameform TGI | Polyglyceryl-3 Diisostearate | 4 |
| Microcrystalline | Microcrystalline Wax | 5.62 |
| SP89 | | |
| Mineral Oil | Mineral Oil | 2.16 |
| Softisan 100 | Hydrogenated Coco-Glycerides | 1.45 |
| Carnauba wax | Copernicia Cerifera (Carnauba) Wax | 1.45 |
| SP63P | | |
| Part 2 | | |
| BYO-I2 | Iron Oxides (C.I. 77492) (And) Isopropyl | 0.33 |
| | Titanium Triisostearate | |
| BRO-I2 | Iron Oxides (C.I. 77491) (And) Isopropyl | 0.33 |
| | Titanium Triisostearate | |
| BBO-I2 | Iron Oxides (C.I. 77499) (And) Isopropyl | 0.1 |
| | Titanium Triisostearate | |
| Part 3 | | |
| SS55M170-CWL5 | Titanium Dioxide | 40 |
| | (And) silica (And) Alumina | |
| | (And) Methicone (And) | |
| | Polyhydroxystearic Acid | |
| | (And) | |
| | Carnauba wax (And) Lauric acid (And) | |
| | Copernicia Cerifera (Carnauba) Wax | |

The crème to powder foundation was prepared as follows: Part 1 was combined in a beaker, stirred and heated to 95° C. The temperature was maintained for 30 minutes. Part 2 was blended together and passed through a micronizer until color was fully dispersed. Then, Part 2 was added to Part 1 and mixed together until homogeneous while maintaining the temperature at 95° C. Next, Part 3 was added to the mixture of Parts 1 and 2 and was homogenized at 4500 rpm for 5 minutes while maintaining the temperature at 95° C. The homogenate was filled at 85° C.

### Example 4

### Preparation of a Sunscreen Composition Incorporating the Novel UV Attenuating Nanoparticles Entrapped in Porous Silica

A sunscreen composition containing porous silica entrapped TiO₂ as in example 1 (to be released under Kobo designation SS55M170-CWL5) was prepared to incorporate the UV attenuating void-filled powder of Example 1. The metal oxide powder was first formulated into a dispersion and was then incorporated into the sunscreen composition. The following ingredients listed in Table 5 were employed in the proportions indicated to prepare the sunscreen composition.

TABLE 5
Sunscreen containing porous silica entrapped TiO2 (SS55M170-CWL5)

| | INCI | % by wt |
|---|---|---|
| Part 1 | | |
| FINSOLV TN | C12-15 Alkyl Benzoate | 21.16 |
| KF-995 | Cyclopentasiloxane | 5 |
| Abil WE 09 | Polyglyceryl-4 Isostearate (And) Cetyl | 5 |
| | Polyglyceryl-4 Isostearate | |
| | (And) Cetyl PEG/PPG- | |
| | 10/1 Dimethicone (And) Hexyl Laurate | |
| White Petrolatum | Petrolatum | 3.5 |
| Silsoft 034 | Caprylyl Methicone | 3 |
| SF96-350 | Dimethicone | 1 |
| Lucentite SAN-P | Lithium Magnesium | 1 |
| | Sodium Silicate (And) | |
| | Distearyldimonium Chloride | |
| Propylene | Propylene Carbonate | 0.1 |
| Carbonate | | |
| Part 2 | | |
| SS55M170-CWL5 | Titanium Dioxide (And) | 10.43 |
| | silica (And) Alumina | |
| | (And) Methicone (And) | |
| | Polyhydroxystearic Acid | |
| | (And) Carnauba wax | |
| | (And) Lauric acid (And) | |
| | Copernicia Cerifera (Carnauba) Wax | |
| Part 3 | | |
| Deionized Water | Water | 43.81 |
| Aculyn 44 | PEG-150/Decyl Alcohol/ | 3.5 |
| | Smdi Copolymer | |
| Sodium Chloride | Sodium Chloride | 1 |
| Germaben II | Propylene Glycol (and) | 1 |
| | Diazolidinyl Urea (and) | |
| | Methylparaben (and) propylparaben | |
| Polysorbate 20 | Polysorbate 20 | 0.5 |

The sunscreen composition was prepared as follows: Part 1 was heated to 50° C. and homogenized at 3,000 rpm until homogeneous. Part 2 was added to Part 1 and homogenized at 4,000 rpm for 5 minutes. Part 3 was heated to 50° C. and was added to Parts 1 and 2 under homogenization at 3,000 rpm for 5 minutes. The homogenate then was cooled to 30° C. in a water bath (with side-sweeping mixing).

While illustrative embodiments have been described above, it is, of course, understood that various modifications will be apparent to those of ordinary skill in the art. Many such modifications are contemplated as being within the spirit and scope of the following claims.

1. A method of producing a substantially non-nano powder composite ingredient for inclusion in cosmetic compositions, comprising:
combining particulates having at least one void therein with UV attenuating nanoparticles so that the UV attenuating nanoparticles enters voids of the particulate;
adding wax at a temperature above the melting point of the wax to the combined nanoparticle-particulate; and
mixing the melted wax with the nanoparticle-particulate to contain the nanoparticles within the at least one void of the particulate to entrap substantially all of the nanoparticles inside the porous particulates.

2. The method of claim 1, wherein the UV attenuating nanoparticles are metal oxides.

3. The method of claim 2, wherein the metal oxides are selected from the group consisting of titanium dioxide, zinc oxide, aluminum oxide, iron oxide, zirconium oxide, chromium oxide, cerium oxide, composites of a metal oxide and composites of a metal oxide and an inorganic salt.

4. The method of claim 3, wherein the metal oxide particles are selected from the group consisting of titanium dioxide and zinc oxide.

5. The method of claim 4, wherein the titanium dioxide or zinc oxide particles are optionally coated with an inorganic coating.

6. The method of claim 5, wherein the inorganic coating is selected from the group consisting of oxides of aluminum, zirconium, silicon, other known inorganic coatings and mixtures thereof before being incorporated into voids of the porous particulates.

7. The method of claim 4, wherein the titanium dioxide or zinc oxide particles are optionally coated with an organic coating.

8. The method of claim 7, wherein the organic coating is selected from the group consisting of silicones, silanes, metal soaps, titanates, organic waxes, amino acids, sodium alginate, polysaccharides, and mixtures thereof.

9. The method of claim 7, wherein said organic coating is optionally applied to the inorganic coating of claim 5.

10. The method of claim 1, further comprising adding a fatty acid.

11. The method of claim 10, wherein the fatty acid is added at a temperature above the melting point of the fatty acid and/or the wax.

12. The method of claim 10, wherein adding a fatty acid includes spraying the fatty acid on the powder.

13. The method of claim 1, further comprising cooling the powder and optionally milling the powder.

14. The method of claim 1, wherein combining the particulate and the UV attenuating nanoparticles includes providing a dispersion of the UV attenuating nanoparticles, adding the dispersion to the particulate, mixing the dispersion and the particulate until generally all the dispersion is absorbed, and optionally removing a solvent contained in the dispersion.

15. The method of claim 1, wherein combining the particulate and the UV attenuating nanoparticles includes blending the particulate as a powder with the UV attenuating nanoparticles until generally all the UV attenuating nanoparticles enter the voids of the particulate.

16. A method of producing a powder, said method comprising the steps of:
(a) dispersing a plurality of UV attenuating nanoparticles in a solvent;
(b) mixing the plurality of dispersed UV attenuating nanoparticles with a plurality of porous particulates having pores so that a plurality of UV attenuating nanoparticles are absorbed in the plurality of porous particulates to form a first powder comprised of a plurality of nanoparticle loaded particulates wherein a plurality of the nanoparticles are in the pores of the porous particles to form a powder comprised of a plurality of nanoparticle-particulate composites;
(c) optionally removing the solvent by heat under vacuum treatment to dry the powder;
(d) repeating steps (b) and (c) to achieve maximum absorption of the plurality of UV attenuating nanoparticles by each of the plurality of porous particulates to produce a dry powder wherein substantially all of the nanoparticles are loaded into one of the plurality of porous particulates; blending a first coating material consisting of a wax at a temperature above the melting point of the wax with the dry powder so that the wax coats and encapsulates each of the plurality of the loaded particulates to entrap substantially all of the nanoparticles into the loaded particulates wherein the sealed nanoparticles are prevented from exiting the particles producing a substantially non-nano powder composite;
(e) adding a second coating material consisting of a fatty acid at a temperature that is above the melting point of the wax and/or the fatty acid to the dry substantially non-nano powder composite or as a solution, a dispersion, a colloid, or a suspension capable of being sprayed onto the composites to form a fatty acid coated composite
(f) cooling the fatty acid coated composite to room temperature; and
(g) optionally milling the fatty acid coated composite into a dry powder.

17. The method of claim 16, wherein the UV attenuating nanoparticles are metal oxides.

18. The method of claim 17, wherein the metal oxides are selected from the group consisting of titanium dioxide, zinc oxide, aluminum oxide, iron oxide, zirconium oxide, chromium oxide, cerium oxide, composites of a metal oxide and composites of a metal oxide and an inorganic salt.

19. The method of claim 18, wherein the metal oxide particles are selected from the group consisting of titanium dioxide and zinc oxide.

20. The method of claim 19, wherein the titanium dioxide or zinc oxide particles are optionally coated with an inorganic coating.

21. The method of claim 20, wherein the inorganic coating is selected from the group consisting of oxides of aluminum, zirconium, silicon, other known inorganic coatings and mixtures thereof before being incorporated into voids of the porous particulates.

22. The method of claim 19, wherein the titanium dioxide or zinc oxide particles are optionally coated with an organic coating.

23. The method of claim 22, wherein the organic coating is selected from the group consisting of silicones, silanes, metal soaps, titanates, organic waxes, amino acids, sodium alginate, polysaccharides, and mixtures thereof.

24. The method of claim 23, wherein said organic coating is optionally applied to the inorganic coating of claim 21.

25. A composition comprising the powder produced according to the method of claim 1.

26. The composition of claim 25, wherein the composite comprises 10-80 wt % of the UV attenuating nanoparticles, 20-80 wt % of the porous particulate, 1-30% wt % of the wax and 1-15 wt % of the fatty acid.
